(19) United States
(12) Patent Application Publication
(10) Pub. No.: US 2025/0322962 A1
(43) Pub. Date: Oct. 16, 2025

(54) Evaluation method of the level of fatigue of an operator and associated evaluation system

(71) Applicant: THALES, Meudon (FR)

(72) Inventors: Lionel GRANIER, MERIGNAC (FR); Florence DURIEUX, MERIGNAC (FR); Philippe DUMERCQ, MERIGNAC (FR)

(21) Appl. No.: 19/171,356

(22) Filed: Apr. 7, 2025

(30) Foreign Application Priority Data
Apr. 12, 2024 (FR) 2403806

(51) Int. Cl. G16H 50/30; A61B 5/00; A61B 5/16; A61B 5/18

(52) U.S. Cl. CPC G16H 50/30; A61B 5/165; A61B 5/18; A61B 5/7221; A61B 5/746; A61B 2503/22

(57) ABSTRACT

A method of evaluation of the level of fatigue of an operator implemented by a transportable evaluation device, the method including identification of the operator, acquisition or determination of contextual data relating to the context in which the evaluation is implemented, acquisition of physiological data of the operator, analysis of all the data acquired or determined for determining the level of fatigue of the operator, and communication of the determined level of fatigue.

### CROSS-REFERENCE TO RELATED APPLICATIONS

This application is a U.S. non-provisional application claiming the benefit of French Application No. 24 03806, filed on Apr. 12, 2024, which is incorporated herein by reference in its entirety.

### TECHNICAL FIELD OF THE INVENTION

The present invention relates to a method of evaluation of a level of fatigue of at least one operator.

The present invention further relates to a system of evaluation of the level of fatigue of the operator which implements the method. The invention relates to the technical field of collecting data

from an operator and determining the fatigue of the operator.

### BACKGROUND OF THE INVENTION

The operator operates e.g. in a critical operational context. In other words, the fatigue of the operator in such critical context can lead to significant consequences. Such is particularly the case in the fields of aeronautics, aerospace, nuclear, medical, etc.

In the aeronautical field, in order to attenuate the risks induced by fatigue, in particular for a pilot in commercial aviation, airlines are putting in place procedures to measure and control the fatigue of the flight personnel (FRMS Fatigue Risk Management System).

The measures concern in particular subjective evaluations of fatigue perceived by operators, in the form of spontaneous self-statements. Such statements are based on personal sensations experienced by operators, that may be biased by cultural, professional or operational factors.

The prior art already proposed a few techniques of evaluation of the fatigue of an operator.

Such solutions are mainly based on biomathematic models that are suitable for interactions in operational use. Same aim in particular to raise the alert if a high level of fatigue is detected and provide limited data on the context of the measures.

Furthermore, such solutions are mainly intended for individual monitoring and do not make possible the centralization and the cross-referencing of data at the scale of a population.

### SUMMARY OF THE INVENTION

The present invention aims to remedy such drawbacks by proposing a solution that makes it possible to objectively evaluate the level of fatigue of an operator, then to identify factors influencing the evolution of their level of fatigue and, more broadly, of a population to which they belong.

To this end, the goal of the invention is to provide a method of evaluation of the level of fatigue of an operator, the method including the following operations implemented by a transportable evaluation device:
identification of the operator;
acquisition or determination of contextual data relating to the context in which the evaluation is implemented;
acquisition of physiological data of the operator;
   the method further including the following operations:
analysis of all the data acquired or determined for determining the level of fatigue of the operator; and
communication of the determined level of fatigue.

The method according to the invention thereby makes it possible not only to objectively evaluate the fatigue of an operator but also to identify, contextualize and centralize the measurements made for the purpose of cross-referencing at the scale of a population.

The invention may thereby be used for the centralized collection and processing of the data on fatigue as well as the contextual data at the scale of a population. As a result, it possible to have a global view of the entire population and thereby to take fatigue into account effectively in order to plan the activities and the missions of the operators.

According to other advantageous embodiments of the invention, the method includes one or a plurality of the following features, taken individually or according to all technically possible combinations:
the operation of identification of the operator includes the implementation of at least one of the following techniques:
   entering a unique personal code;
   reading an external physical or digital medium;
   entering a username or a password;
   recognition of a biometric fingerprint;
contextual data include at least one type of data selected from the group including:
   data relating to the operator's environment;
   operational data relating to activities carried out by the operator;
   physiological data of the operator;
contextual data are entered by the operator and/or acquired from an external device and/or generated by the transportable evaluation device;
the operation of acquisition of the operator's physiological data is implemented from measurements provided by a plurality of sensors integrated into the transportable evaluation device;
the operation of acquisition of physiological data includes at least one of the following sub-operations:
   checking the state of operation of the sensors;
   synchronization of the sensors;
   monitoring of the quality of the measured signal;
   alert in the event of failure of one or a plurality of sensors;
the operation of acquisition of the operator's physiological data is implemented during an activity of the operator other than the interaction with the transportable evaluation device, preferably according to a predetermined minimum duration;
the level of fatigue of the operator is determined by one or a plurality of algorithms for processing physiological data and advantageously contextual data;
the operation of communication of the level of fatigue includes the display of the level of fatigue to the operator and/or the transfer the level of fatigue to an external device;
the method further includes an operation of secure transfer of the acquired and/or determined data to at least one of the elements chosen from the group including:
   an external storage device;
   a mobile device with storage capacity;
   a central server;
   another transportable evaluation device;
the method further includes an operation of analyzing the functioning of the transportable evaluation system including the verification of at least one element chosen from the group including:
   available storage space;
   computing performance;
   state of an internal battery;
   connectivity with a central server and/or other transportable evaluation device;
   operating status of hardware or software components implemented when a malfunction detected;
the method further including an operation of updating at least part of the transportable evaluation device selected from the group including:
   software implemented by the transportable evaluation device;
   data processing algorithms for determining the level of fatigue;
   the nature of contextual data.

A further subject matter of the invention relates to system of evaluation of the level of fatigue of an operator, including means configured for implementing the method as defined hereinabove.

### BRIEF DESCRIPTION OF THE DRAWINGS

The features and advantages of the invention will appear upon reading the following description, given only as an example, but not limited to, and making reference to the enclosed drawings, wherein:

FIG. 1 is a schematic view of an evaluation system according to the invention;

FIG. 2 is a schematic view of a part of a transportable evaluation system which is part of the evaluation system shown in FIG. 1;

FIG. 3 is a flowchart of a detection method according to the invention, the method being implemented by the evaluation shown in FIG. 1; and

FIGS. 4-6 are different illustrations of the implementation of at least certain operations of the method shown in FIG. 4.

### DETAILED DESCRIPTION

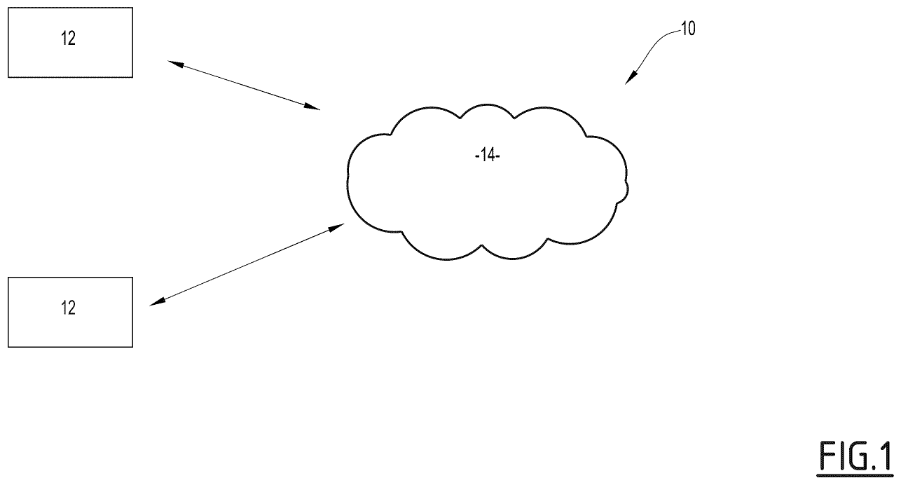

Indeed, FIG. 1 shows a system of evaluation 10 of the level of fatigue of an operator.

Advantageously, the evaluation system 10 may be used in the aeronautical field. In such a case, the operator is part of the flight crew, in particular of the commercial flight crew. In other examples, the operator is one of the flight planning operators or of the maintenance operators or of the aircraft control operators or of the air traffic controllers.

Advantageously, the operator is a pilot apt to pilot an aircraft.

The term “aircraft” refers to any flying craft that may be piloted from the cockpit of the aircraft, as is the case, e.g., with an airplane or helicopter, or else at a distance therefrom, as is the case, e.g., of a drone.

In general, the notion of operator may apply to any other person performing a critical mission, e.g., in the field of transport (e.g., rail or heavy goods vehicles) or in the nuclear or space field, or in medicine.

As indicated hereinabove, the operator performs a mission that is determined by the field of their activity.

More particularly, the mission of the operator includes a plurality of tasks defined according to the skills of the operator.

When the operator is an aircraft pilot, their mission is generally to fly the aircraft from a point of departure to a point of destination.

The evaluation system 10 according to the invention serves to determine the level of fatigue of the operator.

Preferably, the operator is assigned to a mission and the evaluation system determines the operator's level of fatigue before the operator's mission. Therefore, the evaluation method is implemented once before the operator starts their mission.

Preferentially, the evaluation system determines the level of fatigue of the operator before and after the operator's mission. Therefore, the evaluation process is implemented a first time once before the operator starts their mission and once a second time after the operator has completed their mission. For example, both acquisitions are implemented during a pre-mission briefing and post-mission debriefing phase, respectively.

With reference to FIG. 1, the evaluation system 10 includes at least one transportable evaluation device 12 and a central server 14 communicating with the transportable evaluation device 12.

In certain embodiments, the evaluation system 10 includes a plurality of transportable evaluation devices 12. In the example shown in FIG. 1, two transportable evaluation devices 12 are illustrated.

The central server 14 has, e.g., one or a plurality of computers which are suitable for communicating with the or each transportable evaluation device 12 in a secure manner.

The communication may, e.g., be implemented via a direct connection between the server 14 and the or each transportable evaluation device 12 via e.g. one of the wired or wireless interfaces known per se (WIFI, Bluetooth, Ethernet, IR, etc.).

Alternatively, communication may take place via a removable storage medium such as a USB stick, external hard drive or any other mobile device with storage capacity, such as a connected watch.

In another variant, the communication may take place via an Internet connection or any other global or local network.

The central server 14 also has a certain computation capacity used, in particular, for executing applications which are, e.g., stored in the memory thereof.

Finally, the central server 14 has a data storage capacity enabling the data of all the transportable evaluation devices 12 to be collected and kept for a predetermined storage time.

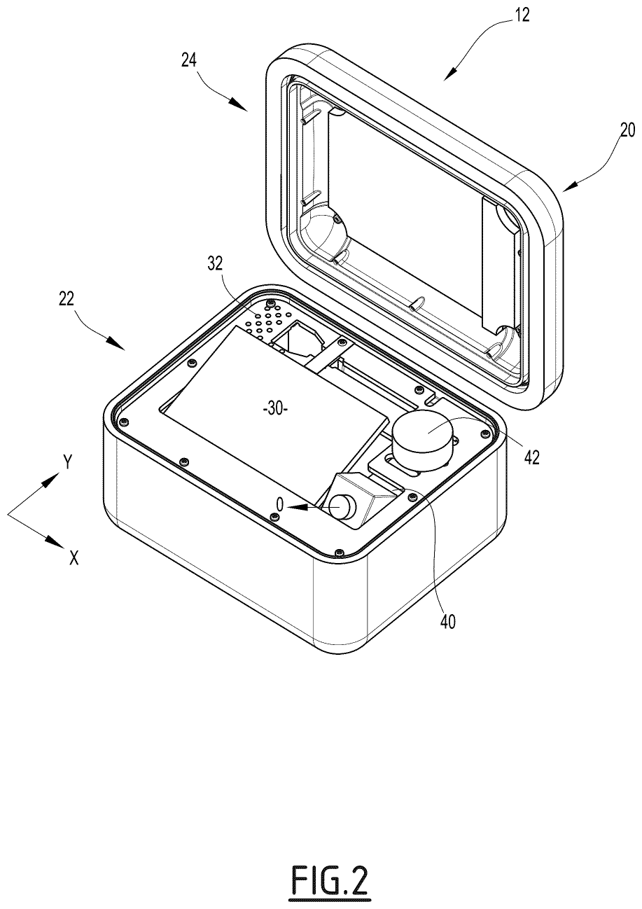

FIG. 2 illustrates in more detail a possible embodiment of a transportable evaluation device 12.

Thereby, and as shown in FIG. 2, the transportable evaluation device 12 has a case 20 integrating different internal components of the transportable evaluation device 12.

More particularly, the case 20 is e.g. in the form of a suitcase or of any other object that may be easily transported. In the example shown in FIG. 2, the case 12 consists of two half-shells 22, 24. The case 20 may also include any other device facilitating the transportation thereof such as e.g. a handle, wheels, etc.

At least one of the half-shells, e.g. the half-shell 24, then forms an opening in the case 20. The half-shell 24 is movable between an extended position and a retracted position. In the open position, illustrated in FIG. 2, the half-shell 24 then provides at least partial access to the internal components of the transportable evaluation device 12.

In general, the case 20 includes a plurality of components accessible by the operator when the half-shell 24 is in its open position and a plurality of components inaccessible by the operator in any position of the half-shell 24.

Among the components accessible by the operator, the transportable evaluation device 12 includes in particular means of interaction with the operator and a plurality of sensors.

The means for interaction with the operator include in particular visual means of interaction such as a screen 30 and auditory means of interaction such as e.g. a loudspeaker 32. The screen 30 and the loudspeaker 32 are, e.g., integrated into an inner surface of the half-shell 22 which is intended to be protected by the half-shell 24 when the latter is in the closed position thereof.

The plurality of sensors includes any sensor serving to acquire the physiological data of the operator.

More particularly, in the example shown in FIG. 2, the plurality of sensors includes a camera 40 configured to acquire images of the operator and a sensor 42 for measuring the heart rate of the operator.

The camera 40 is advantageously oriented toward the operator or has means for orienting same according to the position of the operator.

The sensor 42 for the heart rate of the operator is advantageously removable from the case 20 e.g. to be positioned around an operator's wrist.

To this end, the sensor has 42 e.g. a bracelet that may be attached to the wrist of the operator and a sensitive part which is intended to measure the heart rate of the operator when the bracelet is attached to their wrist.

The measurement of the heart rate is done e.g. by the sensitive part, by the technique called photoplethysmography, called PPG. Alternatively, the sensitive part is configured to carry out the measurement of the heart rate on the basis of an analysis of the electrical response between the wrist of the operator, or by analysis of radar signals propagating through the wrist of the operator.

In certain examples, the sensor 42 is configured to measure other physiological parameters of the operator, such as the blood pressure, the respiration of oxygen, sweating, the level of dehydration.

For the oxygen saturation, the sensor 42 is configured, e.g., to transmit, toward the skin of the operator, and to receive, a light signal including at least two wavelengths. A first wavelength corresponding to a wavelength absorbed by saturated red blood cells, and a second wavelength corresponding to a wavelength absorbed by unsaturated red blood cells. To determine the oxygen saturation, the sensor 42 is then configured to compare the light intensity received in response to each of the two wavelengths.

In general, the sensor 42 may be in the form of a connected watch which may then be stored in the case 20 when need be or worn by the operator, e.g., in order to measure their heart rate.

Of course, the aforementioned functions of the sensor 42 may form separate sensors which may then be arranged in any possible configuration inside the case 20 or else on a surface of the latter.

The non-accessible components of the case 20 are in particular arranged in the inner part of the case 20, e.g., in the inner part of the half-shell 22 and include in particular a calculator, a memory and a power supply module.

The calculator includes in particular a processor for executing a plurality of applications which are stored, e.g., in the memory of the case.

The calculator further includes means of communication with external devices, in particular with other transportable evaluation devices 12 or with the server 14.

The power supply module is used to power all the components of the transportable evaluation device 12. The module includes e.g. a battery for autonomously powering the components. The battery may be associated with a recharging device for connecting the battery to an electrical network in order to recharge same.

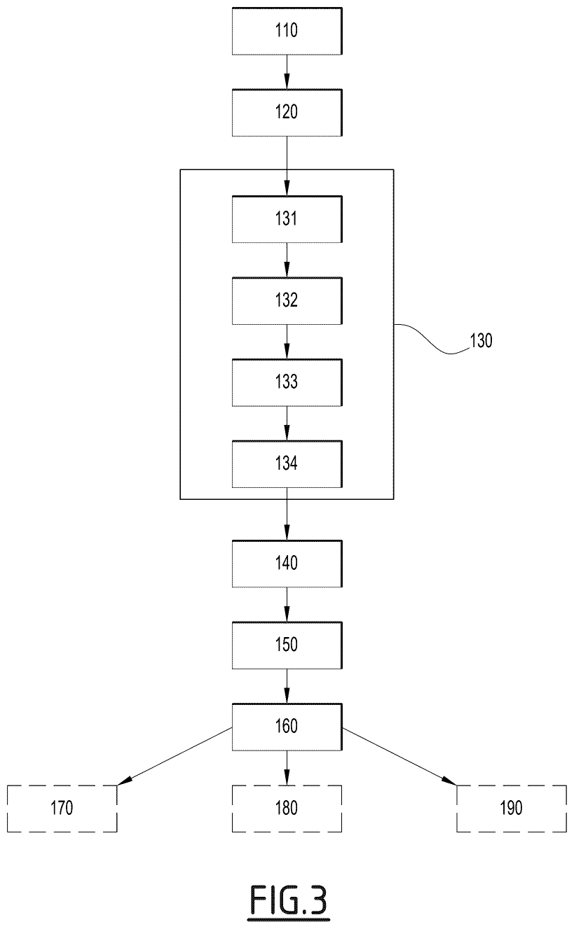

The evaluation device 10 is used to implement the evaluation method according to the invention, which will henceforth be explained with reference to FIG. 3 which shows a flowchart of the operations thereof.

It is considered first of all that the transportable evaluation device 12 is arranged in front of the operator.

When the transportable evaluation device 12 is in the form of a suitcase, the half-shell 24 is then in an open position so that the operator may access the screen 30 and the plurality of sensors of the device 12.

When the sensor 42 is in the form of a watch, the operator then puts same on their wrist.

It is also considered that the camera 40 is oriented toward the operator.

The operation of the device 12 is, e.g., activated by a button or any other control provided for this purpose at the opening of the half-shell 24.

The evaluation method includes an initial operation 110 consisting in identifying the operator by the transportable evaluation device 12.

To this end, and prior to the implementation of the method, the operator is assigned an identification code. The identification code makes it possible to associate the data collected by the device 12 during the following operations, with the given operator. Advantageously, the identification code is associated with the given operator in an anonymized manner. In other words, any information collected and associated with the identification code does not allow the operator to be identified. Alternatively, the collected data is anonymized only before sending the data to the central server 14.

To identify the operator, a plurality of solutions is possible.

According to a first embodiment, the identification of the operator includes the entry of a unique personal code which is then associated with the operator.

The entry may be done via an input interface suitable for such purpose or via a pointing device coupled to a numeric keypad. In order to implement such technique, it is necessary to organize beforehand the allocation of unique identification codes to all operators who may use the transportable evaluation device 12, without keeping track of the allocations for the sake of anonymity of the operators.

According to a second example, the identification of the operator takes place via the reading of an external physical or digital medium. The external medium makes it possible e.g. to keep a unique personal code, encrypted, if appropriate. The conceivable supports are in particular:
QR code if the transportable evaluation device 12 is equipped with a camera with sufficient resolution;
NFC support if the device 12 has a suitable read sensor;
smart card if the device 12 has a suitable means of reading smart cards;
any other removable storage device such as a USB stick, CD, etc. that the device 12 would be able to read.

To implement such solution, it is necessary to organize beforehand, allocating physical or digital media to all operators who may use the device 12 without keeping track of the allocations, in order to preserve the operators' anonymity.

According to a third embodiment, the identification is done through a suitable identifier and a password entered by the operator. Thereof is possible when the device 12 has an input interface or a pointing device coupled to a numeric keypad and a screen.

To implement such solution, it is necessary for each operator to register and then keep the password for their future logins.

In embodiments, the associated identifiers and passwords may be configured beforehand by the device 12 as such. The elements may also be stored on the server 14 and redistributed to the device 12 at the start thereof.

According to a fourth example of embodiment, the identification of the operator takes place via a recognition of a biometric fingerprint.

Same may be a method of fingerprint analysis, of retinal scanning, of face or voice recognition or else of extraction of a distinctive signature from acquired physiological data. The distinctive signature may be formed by a signature of the heart rate, movement, keyboard entry, etc. In such a case, the transportable evaluation device 12 is equipped with one or a plurality of sensors used to recognize such a biometric print.

Of course, the aforementioned examples for the identification of the operator may be combined with each other at least partially to form a secure identification technique preventing any identity theft.

The aforementioned identification techniques may also be reinforced by a multi-factor authentication procedure.

Such multi-factor authentication procedure consists, e.g., in sending a confirmation code to a personal device of the operator having access to the Internet. Such solution may be implemented in particular when the device 12 is as such also connected to the Internet or to any other similar network.

The method further includes an operation 120 during which the transportable evaluation device 12 acquires or determines contextual data.

Contextual data relate to the context in which the evaluation is implemented.

The contextual data may include, e.g., at least one type of data chosen from the group including:
data relating to the operator's environment;
operational data relating to activities carried out by the operator;
physiological data of the operator.

The data relating to the operator's environment may characterize the environment in which the evaluation is carried out. Such data may include, e.g., a time stamp (i.e., time and date) of the evaluation, the geolocation of the location where the evaluation is made, and the temperature and/or humidity and/or brightness and/or noise of the location where the evaluation is made.

Operational data relating to activities carried out by the operator may, e.g., include a characterization of the activities (e.g. the nature of the activities, the duration thereof, etc.), difficulties experienced by the operator during the activities, the organization of the operator's rest periods (e.g., the duration of sleep, the number and duration of naps, the quality of sleep, etc.).

The collection of contextual data may be done in different ways, depending in particular on the nature of the collection.

For example, at least some of the data may be entered directly by the operator, e.g., by means of a pointing device or entry device via filling in the form displayed on the screen 30, or by means of a web page or mobile application associated with the device 12, or by oral answers to questions asked by the device 12. In the latter case, a voice recognition of the answers pronounced by the operator may be used.

At least some contextual data may also be acquired by the sensors of the transportable evaluation device 12 or collected from another system or device.

Thereby, e.g., it is possible to couple a connected watch or share access to a digital agenda, a sports application or a centralization application for health data, to the device 12 in order to collect, e.g., physical activity monitoring data, sleep monitoring data or operator organization assistance.

It is also possible to couple the device 12 to the body in charge of organizing the user's activities to collect data relating to the planning of interventions, flight schedules, etc.

Finally, it is also possible to use a third-party information source such as an API application that presents a software interface for commercial flight tracking or weather reporting.

The method further incudes an operation 130 including the acquisition of physiological data of the operator by the transportable evaluation device 12.

The acquisition is done using the sensors integrated into the transportable evaluation device 12.

In certain embodiments, the operation 130 includes a plurality of sub-operations for ensuring the correct operation of the sensors.

Thereby, e.g., during a first sub-operation 131 which is implemented before physiological data are collected, the device 12 implements a verification of the sensors.

Thereby, e.g., in the case of a sensor that is removable with respect to the device 12, it is recommended to check the pairing thereof with the device 12 as well as the state of the battery thereof. It is also recommended to check the compatibility of the software version with the sensor configuration. A sensor update may then be applied. An obsolete sensor may be inhibited during the measurement. Finally, during the sub-operation 131, the initial state of the sensor may be restored so as not to pollute the acquisition of the current data with data from a previous user of the device 12.

During a subsequent sub-operation 132, the device 12 synchronizes all the sensors with the internal clock thereof so that all the data acquired are time-stamped with the same reference. The device 12 may also check the operating conditions of each sensor and in particular:
the fact that the sensors worn are correctly installed by the user;
the fact that the user adopts the right posture and positions themselves at the right distance from unworn sensors such as the camera 40;
the fact that the sensors are not affected by sources of pollution such as lights, noise, vibration, etc., which may impair the operation thereof.

The interfaces of the device 12 such as the screen 30 and/or the loudspeaker 32 may then be used to accompany the user in the installation of the sensors and to inform them of the progress, success or failure of the installation. The acquisition of physiological data may then take place.

During a sub-operation 133 implemented during, e.g., the acquisition of physiological data, the device 12 implements a monitoring of the quality of the measured signal of each sensor.

More particularly, in such sub-operation, the quality of the signals and the integrity of the sensors are regularly checked in order to inform the user in the event of deterioration of the acquisition conditions such as the position of the sensors worn, the position and the posture of the user, the position of the signals, the malfunction of a sensor, the remaining autonomy, etc.

If a sensor is impacted by external conditions, same may be subject to an audible or visual warning to the operator.

The interfaces of the device 12 also make it possible to inform the operator of the progress of the acquisition of physiological data and to notify them when the data are collected. Such interfaces may also accompany the user in the removal of the sensors and the storing thereof. For example, it should be checked at such stage that battery-powered transducers are powered and the user should be encouraged to replace same on the bases, docking stations or storage cases thereof, if any.

When one or a plurality of sensors are faulty, the acquisition operation 130 includes a sub-operation 134 during which an alert is sent to the operator.

Advantageously, the physiological data acquisition operation 130 is implemented by the device 12 when the operator performs an activity that is different from the interaction with the transportable evaluation device 12.

For example, such operation may be implemented when the operator is debriefing their future activity with colleagues or other operators, or when they doing a regular activity related to their usual tasks.

In other words, the purpose of such operation is to measure the physiological data of the operator in their normal activity.

Also preferably, the physiological data acquisition operation is implemented according to a predetermined minimum duration. Such minimum duration is, e.g., equal to a few minutes, e.g., 5 minutes.

Advantageously, the end of the physiological data acquisition operation may also be signaled by the device 12 via the suitable interfaces.

For example, when the device 12 considers that the physiological data collected are sufficient to assess the level of fatigue of the operator, a corresponding warning may then be issued.

In the example described, the following operations are implemented by the transportable evaluation device 12. However, according to another embodiment, the operations may also be implemented by the central server 14 or else by another transportable evaluation device 12. In such a case, the method further includes an operation of transmission of contextual data acquired or determined during operation 120 as well as of physiological data acquired during operation 130 to the server 14 or to the other transportable evaluation device 12.

The transmission takes place, e.g., in a secure manner.

Also, advantageously according to the invention, before being sent, the data are anonymized if thereof have not been done previously.

In other words, before sending the data, the transportable evaluation device 12 may remove from the data any trace enabling the operator to be identified.

Advantageously, the data are transmitted with an anonymized identifier which makes it possible to determine that the data belong to the same person in order, if appropriate, to complete the data subsequently.

During the next operation 140, the transportable evaluation device 12 analyzes all the data acquired or determined during the preceding operations, in order to determine a level of fatigue of the operator.

To this end, the device 12 implements a plurality of algorithms for processing physiological data and contextual data. According to some embodiments, the algorithms chosen for evaluating the level of fatigue of the operator depend on the nature of the data acquired or determined during the preceding operations.

When such operations is implemented by the transportable evaluation device 12, the level of fatigue is determined, e.g., in real time.

Furthermore, the level of fatigue is advantageously associated with a value taken on a predetermined scale of values.

The predetermined scale may, e.g., vary from 1 to 100 and the determined level of fatigue then takes one of the values in the interval going from 1 to 100.

During the next operation 150, the transportable evaluation device 12 communicates the determined level of fatigue, e.g., to the operator or to any other interested person, such as e.g. a superior of the operator.

The communication incudes, e.g., the display of the determined level of fatigue on the screen 30 of the transportable evaluation device 12 and/or the transfer of the level to an external device.

When the level of fatigue is displayed on the screen 30, same may be the subject of a particular display, e.g., according to the determined level. For example, different classes may be identified on the scale of possible levels of fatigue. When the determined level falls into one of the classes, the display then takes on a form that is associated with said class.

For example, each class may correspond to a moderate, high or very high level of fatigue.

Moreover, when the determined level of fatigue falls into a particular class, e.g., into the class corresponding to the very high level of fatigue, an alert may be raised.

In such a case, operational recommendations or countermeasures to be applied may be displayed to the operator to reduce said level or to prevent certain upcoming tasks.

In the following operation 160, which is implemented when the transportable evaluation device 12 has determined the operator's level of fatigue, same transfers the acquired and/or determined data (including the level of fatigue) to at least one of the elements selected from the group including:
an external storage device;
a mobile device with storage capacity such as a mobile phone or a connected watch;
the remote server 14;
another transportable evaluation device 12.

The transfer performed by the device 12 is supervised and the operator may be informed of the progress, failure and success thereof.

If a datum is correctly transferred, same is advantageously deleted from the transportable evaluation device 12. In the event of a transfer failure, the datum is kept for a subsequent transfer attempt. In case of consecutive failures, an alternative transfer mode may be applied.

Preferably, the transfer is carried out in real time, immediately after the end of the collection and determination of the corresponding data. The transfer may be triggered automatically or manually.

When the transfer takes place toward the central server 14, it is appropriate to secure the transfer in a particular manner.

For example, a first countermeasure applicable to secure the transfer is the filtering by IP address, which only authorizes the transfer from previously authorized connections. Such solution is easy to implement.

A second countermeasure is to protect the transfer by authentication. If the transfer takes place in real time or immediately after the collection and determination of the corresponding data, the authentication rests on the password provided by the user. If the transfer is triggered automatically during a period of inactivity of the device 12, the authentication rests on the password stored on the device.

The method may further include an optional operation 170 of analyzing the operation of the transportable evaluation device 12. The analysis is, e.g., implemented by the device 12 as such or by the central server 14 or another transportable evaluation device 12.

The operation 170 is, e.g., implemented when the device 12 is not used by the operator, e.g., after the level of fatigue of the operator has been determined. Such operation makes it possible to identify failure cases or anticipate the maintenance needs of the device 12.

More particularly, during such operation, the verification of at least one element chosen from the following group may be performed:
available storage space
calculation performance (duration, precision, relevance of the result);
sensor integrity (failure case, loss of autonomy of the battery);
duration of data transfers;
state of an internal battery;
connectivity to the central server 14 or other transportable evaluation device 12.

Following the implementation of the analysis operation, maintenance actions may be carried out if need be. For example, a specific intervention on a system or a generic scheduled maintenance action (e.g., deleting the oldest data stored on the device if the storage space is limited) may be involved.

In certain embodiments, the method further includes an operation 180 of updating at least a portion of the transportable evaluation device 12. Such part is advantageously chosen from the group including:
software implemented by the device 12;
data processing algorithms to determine the level of fatigue;
the nature of contextual data.

For example, the update may adapt the nature of the contextual data acquired or determined during operation 120.

Furthermore, in the case of a large number of transportable evaluation devices 12 deployed at different operational sites, an automated update distribution solution should be implemented with minimal impact on the availability of the devices. Furthermore, because devices may cover different use cases, it is necessary to master the software version applied and the deployment window of the updates.

In such a case, e.g. an OTA (over the air) update may be done according to the following sub-operations:
deployment of software components on a distribution server (e.g. the central server 14);
definition of a subset of versioned software components defining a consistent application delivery;
testing the application delivery in a validation environment;
assignment of the application delivery to the subset of devices 12 targeted by the deployment of the delivery;
the device 12 regularly and spontaneously checks the availability of a new application delivery to be installed with the distribution server;
the device 12 downloads the exhaustive list of software components and the respective versions thereof to be installed from the distribution server;
the device 12 archives the current version of each component to be updated, downloads and then installs the new version;
the device 12 notifies the distribution server of the progress of the installation; and
in case of failure to install one of the components, the update is canceled and a return to the latest stable application version is performed.

In addition, a deployment control interface may be coupled to the distribution server. It is thereby possible to make sure about the version currently installed on each system deployed on an operational site.

In certain embodiments, the method may also include an operation 190 of analysis of the use of the device 12. Such operation is implemented for analyzing the behavior of the operator in relation to the device, in order to make the use of the device more fluid and faster and also to improve the relevance of the collected data.

More particularly, by observing the modes of use of the interfaces and the time required at each operation of the data collection, it is possible to identify the operations that are the most tedious for the operator. The operations thereby identified are then improved through an update.

The analysis may be done e.g. in three sub-operations:
each action on an interface of the device 12 generates a trace;
the traces are transferred to the central server 14; and
centralized data are cross-referenced in order to identify common difficulties of use.

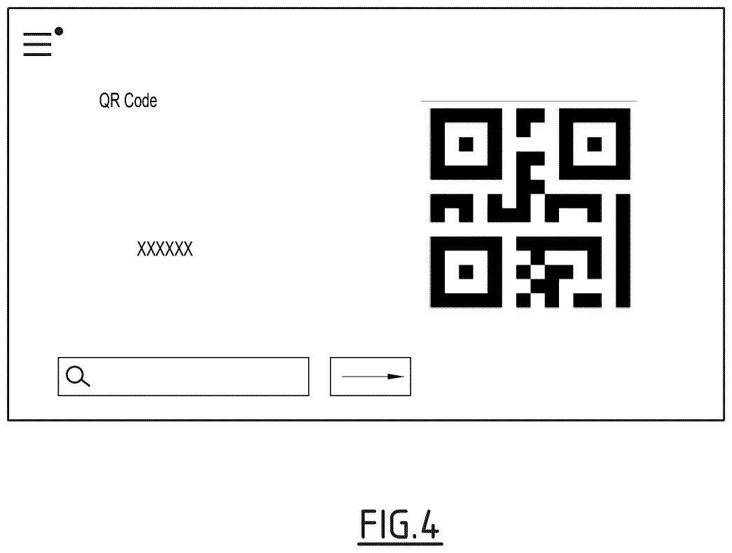

FIGS. 4-6 illustrate the result of implementing at least some of the operations of the method explained hereinabove.

Thereby, e.g., FIG. 4 illustrates an example of the implementation of operation 110 of the identification of the operator.

According to such example, the operator presents e.g. a QR code on a physical support in front of the camera 40 of the device 12. The QR code is then recognized and identifies the operator. In such a case, the display on the screen 30 may be similar to FIG. 4.

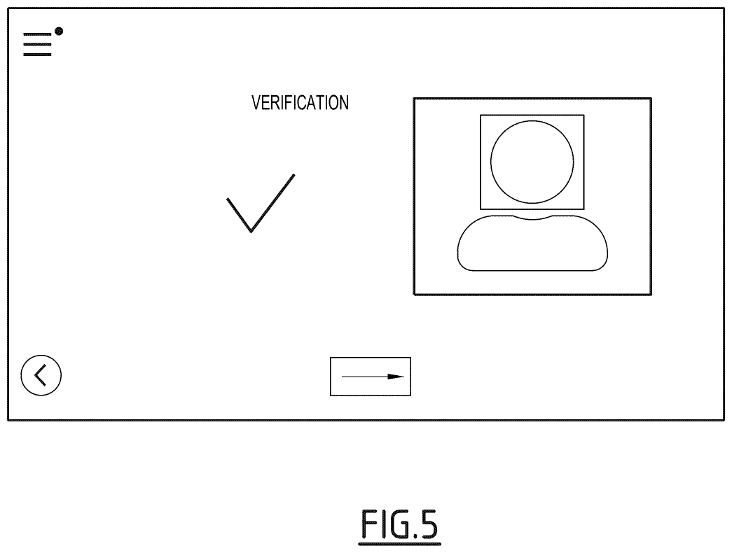

FIG. 5 illustrates an example of the implementation of the operation 130 of acquisition of the physiological data of the operator.

According to such example, the device 12 verifies the positioning of the operator in front of the camera 40.

When the positioning is correct, the screen 30 may display an image similar to the image of FIG. 5.

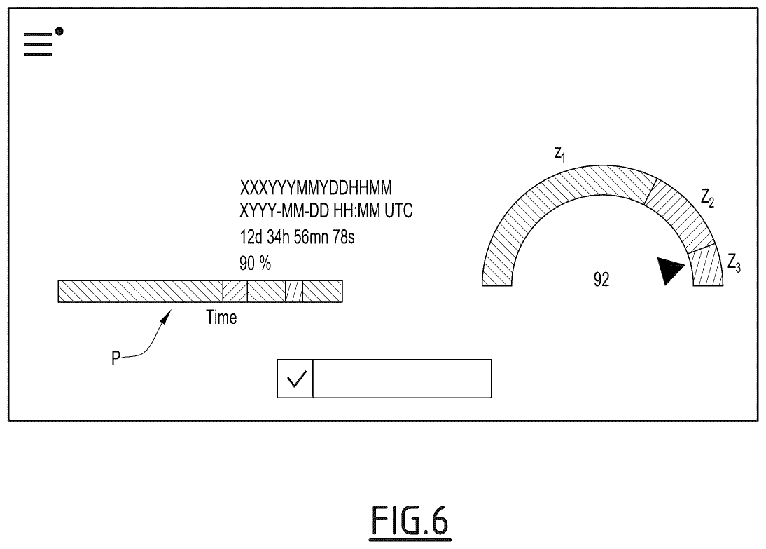

Finally, FIG. 6 illustrates an example of the implementation of operation 150 of communication of the determined level of fatigue to the operator.

According to said figure, three classes of fatigue are determined:
moderate, corresponding to zone Z1 in FIG. 6;
high, corresponding to zone Z2 in FIG. 6; and
very high, corresponding to the zone Z3 in FIG. 6.

The determined level of fatigue may also be displayed (92 in the example in FIG. 6) with an indication of the class to which same belongs.

In this way, it may be understood that the present invention has a certain number of advantages.

More particularly, the invention makes possible the collection of necessary and sufficient data for the measurement of a physiological state and the identification of possible external factors favoring or limiting said state.

The method according to the invention is scalable, adjustable to different systems and each operation presented may be implemented by different techniques.

It should also be understood that the level of fatigue determined by such method should be understood broadly and further includes other physiological states such as the level of stress, the level of mental load, a measurement of physical performance, etc.

Of course, other embodiments of the invention are further possible.

1. A method of evaluating a level of fatigue of an operator, implemented by a transportable evaluation device, the method comprising:
identifying an operator;
acquiring or determining contextual data relating to the context in which the evaluation is implemented;
acquiring physiological data of the operator;
analyzing the contextual data to determine a level of fatigue of the operator; and
communicating the determined level of fatigue.

2. The method according to claim 1, wherein said identifying comprises at least one of:
entering a unique personal code,
reading an external physical or digital medium,
entering a username or a password, and
recognizing a biometric fingerprint.

3. The method according to claim 1, wherein the physiological data comprises at least one type of data selected from:
data relating to the operator's environment; and
operational data relating to activities carried out by the operator.

4. The method according to claim 1, wherein the contextual data are entered by the operator and/or acquired from an external device and/or generated by the transportable evaluation device.

5. The method according to claim 1, wherein said acquiring physiological data is implemented from measurements provided by a plurality of sensors integrated with the transportable evaluation device.

6. The method according to claim 5, wherein said acquiring physiological data comprises at least one of:
checking a state of operation of the sensors;
synchronizing the sensors;
monitoring quality of a measured signal; and
alerting in the event of failure of one or a plurality of sensors.

7. The method according to claim 1, wherein said acquiring physiological data is implemented during an activity of the operator other than interaction with the transportable evaluation device.

8. The method according to claim 1, wherein said acquiring physiological data is implemented during an activity of the operator other than interaction with the transportable evaluation device according to a predetermined minimum duration.

9. The method according to claim 1, wherein said analyzing determines the level of fatigue of the operator by one or a plurality of algorithms for processing physiological data.

10. The method according to claim 1, wherein said analyzing determines the level of fatigue of the operator by one or a plurality of algorithms for processing physiological data and contextual data.

11. The method according to claim 1, wherein said communicating comprises displaying the level of fatigue to the operator and/or transferring the level of fatigue to an external device.

12. The method according to claim 1, further comprising securely transferring the contextual data to at least one of:
an external storage device;
a mobile device with storage capacity;
a central server; and
another transportable evaluation device.

13. The method according to claim 1, further comprising analyzing functioning of the transportable evaluation device comprising verifying at least one of:
available storage space;
computing performance;
state of an internal battery;
connectivity with a central server and/or other transportable evaluation device; and
operating status of hardware or software components implemented when a malfunction is detected.

14. The method according to claim 1, further comprising updating at least part of the transportable evaluation device selected from:
software implemented by the transportable evaluation device;
data processing algorithms for determining the level of fatigue; and
the nature of the contextual data.

15. The method according to claim 1, wherein the operator is assigned to a mission and wherein the method is carried out before the mission of the operator.

16. The method according to claim 1, implemented when the operator debriefs his future mission or carries out a usual activity relating to his usual tasks which are distinct from his mission.

17. A system for evaluating a level of fatigue of an operator, comprising modules configured to implement the method according to claim 1.
